(19) United States
(12) Patent Application Publication
(10) Pub. No.: US 2023/0227775 A1
(43) Pub. Date: Jul. 20, 2023

(54) METHOD OF PREPARING FEEDER CELL CONCENTRATES

(71) Applicant: Oklahoma Blood Institute, Oklahoma City, OK (US)

(72) Inventors: Charles Mooney, Oklahoma City, OK (US); Jennifer Chain, Oklahoma City, OK (US)

(21) Appl. No.: 18/095,042

(22) Filed: Jan. 10, 2023

Related U.S. Application Data
(60) Provisional application No. 63/300,355, filed on Jan. 18, 2022.

(51) Int. Cl. C12N 5/078; C12N 13/00; A01N 1/02; B01D 21/26

(52) U.S. Cl. CPC C12N 5/0634; C12N 13/00; A01N 1/0284; B01D 21/262

(57) ABSTRACT

Feeder cell products are needed to enhance growth of cells that are being cultured. The current process produces feeder cells by selecting a source for blood material containing mononuclear cells (MNCs) and other blood components, combining blood material from a plurality of donors, isolating the MNCs from the other blood components so as to form isolated heterogenous MNCs. Afterwards, the isolated heterogeneous MNCs is irradiated to produce the feeder cells.

### CROSS REFERENCE TO RELATED APPLICATION

This application claims the benefit of U.S. Provisional Patent Application No. 63/300,355 filed on Jan. 18, 2022, which is hereby incorporated by reference.

### FIELD

The present invention is a method for preparing pools of mononuclear cells (MNCs) that can be used to enhance the expansion of cells when cultured.

### BACKGROUND

Human blood has four primary components: red blood cells (RBC), white blood cells (WBC), platelets and plasma. Each blood cell is derived from hematopoietic stem cells. These cells mature into either a myeloid or lymphoid cell line (FIG. 1). Except for RBC's, all human blood cells have a nucleus. Cells that have only one nucleus are called mononuclear cells (MNCs) and are a fraction of WBCs. All the cells in the lymphoid cell line are MNCs and are currently being used in immune cell-based therapies. Lymphocytes and monocytes are subtypes of MNCs. Scientists are genetically modifying monocytes, T lymphocytes, and natural killer cells and using them to treat cancers. The field of science that uses genetically modified immune cells to make cancer drugs is commonly called cell therapy.

Most cell therapies used to treat cancers are gene-modified immune cells that have been reprogrammed to attack a specific molecule on the cancer cell. Once the immune cells have been reprogrammed, they are expanded to generate enough cells for an effective cancer treatment. The expansion process creates controlled conditions that mimic the natural environment of the immune cells and provides specific nutrients for the cells to use to create many more of themselves. This expansion process is generally termed “cell culture” or “tissue culture.”

Optimal cell culture technique requires knowing the exact nutrients each cell type needs and mimicking that natural environment. Immune cells grow better in the presence of other immune cells, so mixing MNCs with gene-modified T lymphocytes creates an ideal culture environment. However, it is important that only the gene modified cells expand. Therefore, it is preferred for the MNCs to be non-reproducing while remaining alive and biologically active. Such biologically active but growth-arrested cells are typically referred to as “feeder cells”. Feeder cells have been shown to significantly enhance the growth of certain cell types.

Accordingly, it would be advantageous to have a process that can produce this type of feeder cell.

### SUMMARY

The present disclosure provides a method of preparing heterogeneous, irradiated, MNCs for use as feeder cells.

This method uses MNCs isolated from blood material from sources, such as either apheresis platelet products (for example Leukoreduction System (LRS) chambers) or whole blood-derived buffy coats (WB buffy coats).

Blood material from multiple donors are pooled into a single container to create a heterogenous population of cells.

The pooled heterogenous population of cells can be processed with density gradient medium (such as a Ficoll-Paque density gradient medium) to isolate the MNC fraction from the pooled material.

Isolated MNCs can be washed and have its concentration adjusted to a desired or predetermined number of MNCs per mL.

The isolated MNCs, typically after washing and adjusting concentration, can be exposed to an adsorbed radiation sufficient to inhibit cell division and growth while still maintaining biological activity, for example ≥2500 rads of radiation.

A cryopreservative can be added to the irradiated MNCs to allow the irradiated MNCs to be frozen.

Pooled, isolated, irradiated, and aliquoted MNCs can be cryopreserved then thawed for use as feeder cells for cell cultures.

### BRIEF DESCRIPTION OF DRAWINGS

FIG. 1 is a diagram of hematopoiesis (also referred to as hemopoiesis) which is the formation of blood cellular components. FIG. 1 illustrates various blood components formed during hematopoiesis.

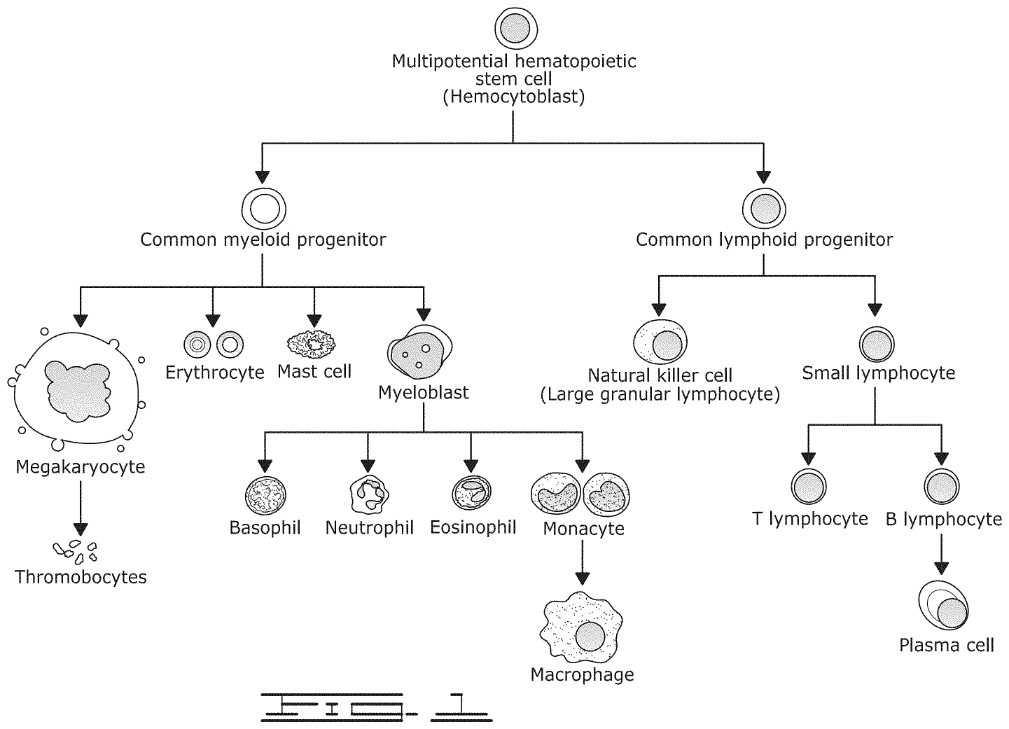

FIG. 2 is a diagram of the separation of whole blood into plasma, buffy coat and red blood cells.

FIG. 3 is a process flow chart of a process for manufacturing feeder cell concentrates in accordance with this disclosure.

FIG. 4 is a schematic of conceptualized diagram of the process of preparing feeder cell concentrates in accordance with the flow chart of FIG. 3.

### DETAILED DESCRIPTION

The present disclosure may be understood more readily by reference to these detailed descriptions. Numerous specific details are set forth to provide a thorough understanding of the various embodiments described herein. However, it will be understood by those of ordinary skill in the art that the embodiments described herein and can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Before discussing the presently disclosed inventive concepts in detail by way of exemplary description and drawings, it is to be understood that the inventive concepts disclosed herein are not limited in application to the details of construction and the arrangement of the compositions, formulations, steps, or components set forth in the following description or illustrated in the drawings. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting except where indicated as such.

All of the compositions, devices, systems, and/or methods disclosed herein can be made and executed without undue experimentation in light of the present disclosure. Although certain steps are described herein and illustrated in the figures as occurring sequentially, some steps may occur simultaneously with each other or in an order that is not depicted.

As utilized in accordance with the present disclosure, the following terms, unless otherwise indicated, shall be understood to have the following meanings: the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements, or method steps.

Throughout this disclosure, the terms “about”, “approximate”, and variations thereof, are used to indicate that a value includes the inherent variation or error for the device, system, the method being employed to determine the value, or the variation that exists among the donors.

The present disclosure is directed at a process of harvesting and pooling mononuclear cells (MNCs) separated from red blood cells (RBCs) and other white blood cells (WBC) that have been salvaged from apheresis platelet products or whole blood (WB) buffy coat to create a heterogeneous concentrate of MNCs. Then, exposing these MNCs to radiation creates a feeder cell concentrate that can be used to accelerate the growth of genetically modified cells in cultures.

As indicated above, immune cells grow better in the presence of other immune cells, so mixing MNCs with gene-modified T lymphocytes creates an ideal culture environment. However, it is important that only the gene modified cells expand. Therefore, the non-gene modified cells are pre-treated with radiation to make them incapable of expanding while remaining alive and biologically active; hence, producing MNC feeder cells. These feeder cells produce essential factors that support the growth of the surrounding cells they are mixed with and have been shown to significantly enhance the growth of certain cell types.

The feeder cells produced herein do not come from the same donor as the gene-modified cells they are supporting. This heterogeneity is advantageous. First, the mis-matched surface molecules of the feeder cells stimulate the gene-modified cells to divide. Also, there can be variations in the production and concentration of growth factors by cells from person to person, therefore, it is beneficial to have the feeder cells composed of cells from multiple donors.

As discussed above, it is beneficial to have the feeder cells composed of cells from multiple donors. The present process advantageously can produce this type of feeder cells by pooling MNCs harvested from donated whole blood and/or Leukoreduction System (LRS) chambers after a platelet apheresis procedure. Blood centers have large numbers of MNC-containing blood products available to create high concentrations of heterogeneous, irradiated MNCs for the use of enhancing the growth of gene-modified human cells.

One source of the MNCs is harvesting from LRS chamber after a platelet apheresis procedure. During the process of performing platelet apheresis procedures on Trima™ cell separation devices, WBCs are removed from the platelet product collected from a blood donor. The WBCs are removed by a process proprietary to Terumo BCT, Inc, 10811 Collins Ave, Lakewood, Colo. 80215. This process uses the company's patented Leukoreduction System (LRS). The LRS chamber is a cone shaped chamber integrated into the plateletpheresis collection kit.

After the platelet apheresis procedure is complete, the collection kit is removed from the collection device and discarded as medical waste. However, the LRS chamber can be salvaged from the medical waste and its contents removed. The contents of the LRS chamber includes a high concentration of MNCs with a low concentration of red blood cells, granulocytes and platelets. For example, typical LRS chambers contain approximately 1-3×10⁹ WBCs, of which >95% are MNCs and <5% are granulocytes, on average. In addition, each LRS chamber contains approximately 30-60×10⁹ RBCs and 1.0-5.0×10⁹ platelets in a total volume of 8-10 mL of whole blood.

As will be further discussed below, the contents of many LRS chambers can be combined into one pool as a source of heterogenous MNCs. Thus, the contents of LRS chambers from different donors are pooled as a single source of heterogenous MNCs.

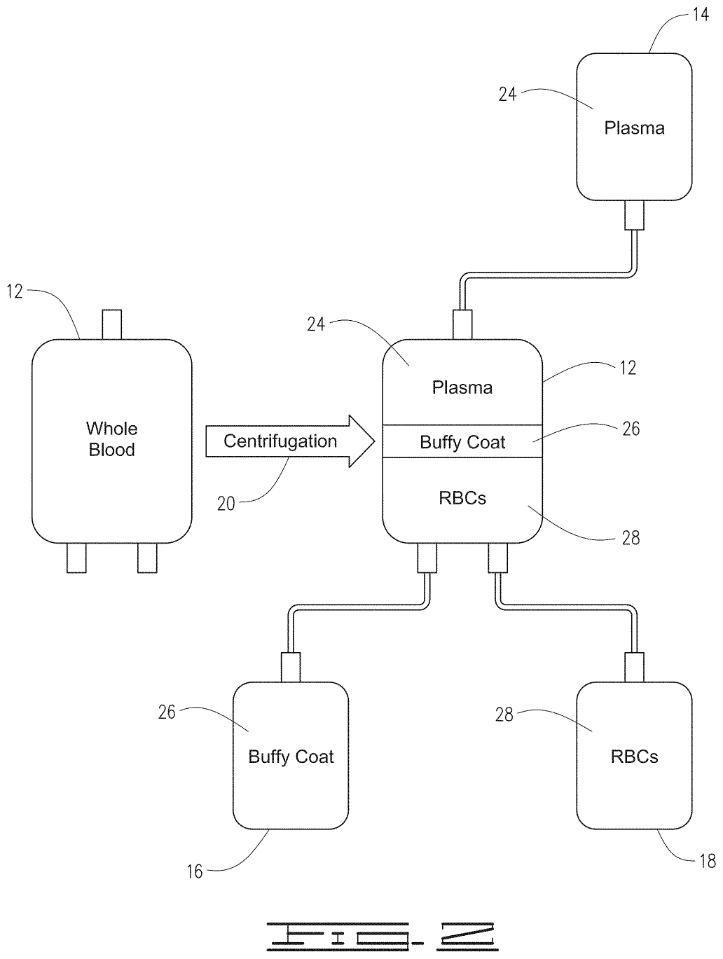

Another source of the MNCs is buffy coat derived from donated WB. With reference to FIG. 2, WB is collected into a commercially available blood pack, containing a collection bag 12 and multiple bags 14, 16 and 18 that can be used to separate different components of the WB. The collection bag 12 typically collects from 450 mL to 500 mL of WB.

After the blood is collected in bag 12, The collection bag containing the WB is centrifuged 20. After centrifugation, the WB unit is separated into three layers based on the molecular weight of each layer: plasma layer 24, buffy coat layer 26 and RBCs layer 28. Pressure is applied to the layered collection bag. A seal between one of the attached empty bags is broken and the top layer of plasma 24 is pushed out of the collection bag and into the empty bag 14. Similarly, the layer of RBCs 28 are transferred to bag 18 and the layer is the buffy coat 26 is transferred to bag 16. Buffy Coat is the buff colored layer that contains WBCs and platelets. Thus, a seal between another empty bag is broken and the buffy coat/WBC layer is pushed from the collection bag into bag 16. The WBCs in this bag are used as a source of MNCs for this disclosure.

As will be further discussed below, the contents of many buffy coat bags 16 can be combined into one pool as a source of heterogenous MNCs. Thus, the buffy coat bags from different donors are pooled as a single source of heterogenous MNCs.

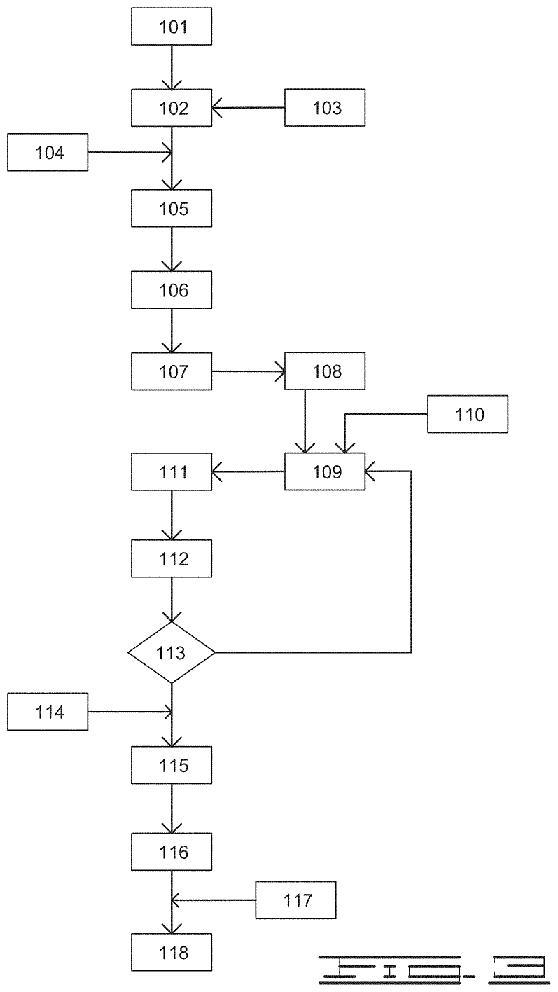

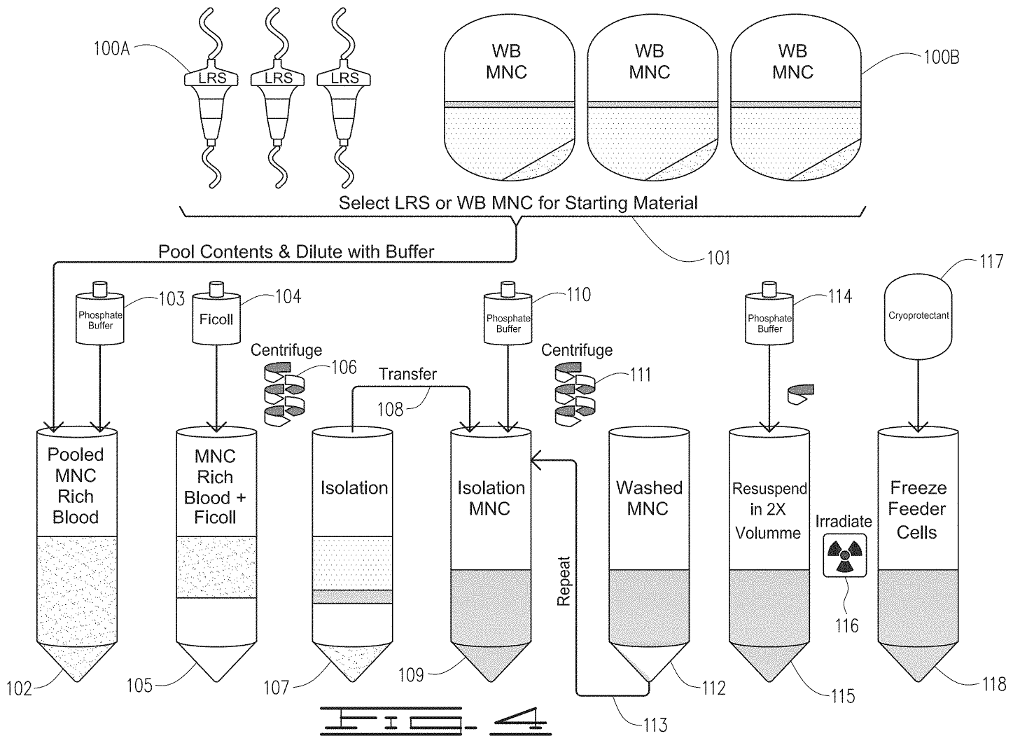

Turning now to FIGS. 3 and 4, a process for manufacturing the feeder cell concentrates is illustrated. FIG. 3 illustrates the steps in simplified step-process flow chart, and FIG. 4 illustrates the same process in a conceptualized diagram. As illustrated, the first step 101 for manufacturing feeder cell concentrates is to select a source of blood material. As described above the source of blood material contains MNCs and other blood components. The blood material source can be either MNCs harvested from LRS chamber after a platelet apheresis procedure (100-A) or buffy coat derived from donated WB (100-B).

After MNC source 1 (100-A, typically containing approximately 1-3×10⁹ MNCs) or MNCs source 2 (100-B, typically containing 0.5-1.0×10) is selected, either multiple units of source 1 (the contents of a plurality of LRS chambers) or source 2 (contents of a plurality of buffy coat bags 16) are pooled into a single container. (Step 102) In this manner, a plurality of the blood material is combined to produce a heterogeneous material, wherein the heterogenous material is pooled in that there is blood material from a plurality of donors in the pooled material.

Next at step 103, a buffer solution, such as a phosphate buffer solution (PBS), is added to dilute the MNC source pool, or heterogenous material. For example, the heterogenous material can be diluted to produce about a 1:1-rich pool.

Next the MNCs are isolated from the other blood components in the heterogenous material so as to form isolated heterogenous MNCs. For example, the MNCs are isolated from the blood components (other cells) by using a commercially available density gradient medium, typically a Ficoll-Paque density gradient medium (Ficoll), such as that offered by Miltenyi Biotec. (Step 104). The MNC-rich pool is layered onto the Ficoll (Step 105) and centrifuged (Step 106), typically at approximately 2000 RPM though other suitable rpms can be used as long as they achieve separation of the MNCs and other blood components. Centrifugation separates the MNC from other blood components, such as RBC/granulocytes and plasma. After centrifugation the blood is layered into different blood components. (Step 107) The top layer is plasma, the next layer is MNCs, under the MNCs is Ficoll. The layer below the Ficoll is WBCs and RBCs. The interface between the Ficoll layer and plasma contains the MNCs. The MNC layer can be removed from the interface, such as by using a sterile pipet. (Step 108)

After the MNC layer has been isolated (109), further buffer solution (such as PBS—Step 110) and centrifugation (Step 111) are used to wash the isolated MNCs and remove further other blood components to produce a washed MNC (Step 112), which is a washed heterogenous MNCs. If further washing is needed, the wash step can be repeated (Step 113). Typically, there will be two such washings (buffer solution and centrifugation).

A sample is obtained and the total nucleated cell count is performed. Where two wash steps are performed, the sampling and count is generally performed between the two wash steps. Using the total nucleated cell counts, a volume of buffer solution (or PBS) is added (114) to the isolated and washed heterogenous MNCs to create the desired (predetermined) MNC per mL concentration (Step 115). Generally, the predetermined concentration will be about twice the desired final concentration—that is, the predetermined concentration for the resulting product to be used as feeder cells for a culture. For example, the predetermined concentration typically is about 1-4×10⁸ per ml)

The washed heterogenous MNCs are irradiated (step 116). While irradiation can be before adjusting the MNCs concentration as described above, typically the irradiation is after concentration adjustment. For example, the washed heterogenous MNCs are exposed to a radiation source to deliver a dose of radiation to the washed heterogenous MNCs effective and sufficient to inhibit cell division and growth while still maintaining biological activity. Generally, over 2500 rad of radiation can be used, and more typically from 2500 rads to 75,000 rads of radiation can be used. For example, about 50,000 rads can be used.

After concentration adjustment and radiation, the resulting heterogenous MNCs are now the feeder cell product. For storage before use, the feeder cell product can be frozen. For example, a cryopreservation solution (such as dimethylsulfixude solutions offered by CryoPur under the trademark CryoPur) can be added to the irradiated MNCs. (Step 117) Next the feeder cells (irradiated MNCs) are cooled to between −20° C. to −90° C. (Step 118) Although not depicted in the illustration, step 117 and step 118 are typically performed in more than one container. The number of containers needed is determined by the total nucleated cell count and the final cell concentration desired in each container.

The pooled, washed, irradiated, cryopreserved MNCs are stored between −20 to −80° C. The frozen MNC units can be thawed by the user and used as feeder cells for cell cultures.

Embodiments of the invention can be further understood by the following numbered paragraphs.

1. A process for producing feeder cells, the process comprising: selecting a source for blood material containing mononuclear cells (MNCs) and other blood components;

combining a plurality of the blood material to produce a heterogeneous material, wherein the heterogenous material is pooled in that there is blood material from more than one donor in the pooled material;

isolating the MNCs from the other blood components so as to form isolated heterogenous MNCs;

radiating the isolated heterogeneous MNCs such that there is an adsorbed radiation dose sufficient to inhibit cell division and growth while still maintaining biological activity to thus produce the feeder cells.

2. The process of paragraph 1, wherein the source is selected from apheresis platelet products or whole blood (WB) buffy coat, and mixtures thereof.

3. The process of paragraphs 1 or 2, wherein prior to the step of isolating the MNCs, a buffer solution is added to dilute the heterogenous material.

4. The process of any preceding paragraph, wherein the step of isolating includes:

centrifuging the heterogenous material such that the MNCs are separated from the other blood components so as to produce an MNCs layer and one or more other layers; and

isolating the MNCs layer from the other layers;

5. The process of paragraph 4, wherein prior to the step of centrifuging, a density gradient medium is added to the source of MNCs

6. The process of any preceding paragraph, further comprising, before the step of radiating:

washing the isolated heterogeneous MNCs; and

adjusting the MNCs concentration of the isolated heterogenous MNCs.

7. The process of paragraph 6, wherein the step of washing comprises mixing a buffer solution with the isolated heterogenous MNCs, and centrifuging the resulting mixture.

8. The process of paragraph 7, wherein the step of washing is repeated.

9. The process of any preceding paragraph, wherein the absorbed radiation dose is from 2,500 to 75,000 rads.

10. The process of any preceding paragraph, further comprising, after the step of radiating, storing the feeder cells at temperature between −20° C. to −90° C.

What is claimed is:

1. A process for producing feeder cells, the process comprising:
selecting a source for blood material containing mononuclear cells (MNCs) and other blood components;
combining a plurality of the blood material to produce a heterogeneous material, wherein the heterogenous material is pooled in that there is blood material from a plurality of donors in the pooled material;
isolating the MNCs from the other blood components so as to form isolated heterogenous MNCs;
radiating the isolated heterogeneous MNCs such that there is an adsorbed radiation dose sufficient to inhibit cell division and growth while still maintaining biological activity to thus produce the feeder cells.

2. The process of claim 1, wherein the source is selected from apheresis platelet products or whole blood (WB) buffy coat, and mixtures thereof

3. The process of claim 1, wherein prior to the step of isolating the MNCs, a buffer solution is added to dilute the heterogenous material.

4. The process of claim 1, wherein the step of isolating includes:
centrifuging the heterogeneous material such that the MNCs are separated from the other blood components so as to produce an MNCs layer and one or more other layers; and
isolating the MNCs layer from the other layers.

5. The process of claim 4, wherein prior to the step of centrifuging, a density gradient medium is added to the source of MNCs.

6. The process of claim 1, further comprising, before the step of radiating:
washing the isolated heterogeneous MNCs; and
adjusting the MNCs concentration of the isolated heterogeneous MNCs.

7. The process of claim 6, wherein the step of washing comprises mixing a buffer solution with the isolated heterogenous MNCs, and centrifuging the resulting mixture.

8. The process of claim 7, wherein the step of washing is repeated.

9. The process of claim 8, wherein the source is selected from apheresis platelet products or whole blood (WB) buffy coat, and mixtures thereof

10. The process of claim 9, wherein prior to the step of isolating the MNCs, a buffer solution is added to dilute the heterogenous material.

11. The process of claim 10, wherein the step of isolating includes:
centrifuging the heterogeneous material such that the MNCs are separated from the other blood components so as to produce an MNCs layer and one or more other layers; and
isolating the MNCs layer from the other layers.

12. The process of claim 11, wherein prior to the step of centrifuging, a density gradient medium is added to the source of MNCs.

13. The process of claim 1, wherein the absorbed radiation dose is from 2,500 to 75,000 rads.

14. The process of claim 1, further comprising, after the step of radiating, storing the feeder cells at temperature between −20° C. to −90° C.

15. A process for producing feeder cells, the process comprising:
selecting a source for blood material containing mononuclear cells (MNCs) and other blood components, wherein the source is selected from apheresis platelet products or whole blood (WB) buffy coat, and mixtures thereof;
combining a plurality of the blood material to produce a heterogeneous material, wherein the heterogenous material is pooled in that there is blood material from a plurality of donors in the heterogeneous material;
adding a buffer solution to dilute the heterogeneous material;
adding a density gradient medium to the heterogenous material;
centrifuging the heterogeneous material such that the MNCs are separated from the other blood components so as to produce an MNCs layer and one or more other layers;
isolating the MNCs layer from the other layers so as to isolate the MNCs from the other blood components and form isolated heterogenous MNCs;
washing the isolated heterogeneous MNCs by mixing a buffer solution with the isolated heterogenous MNCs, and centrifuging the resulting mixture to produce washed heterogenous MNCs;
adjusting the MNCs concentration of the washed heterogeneous MNCs;
subsequent to adjusting the MNCs concertation, radiating the washed heterogeneous MNCs such that there is an adsorbed radiation dose sufficient to inhibit cell division and growth while still maintaining biological activity to thus produce the feeder cells, and wherein the adsorbed radiation does is from 2,500 to 75,000 rads; and
storing the feeder cells at a temperature between −20° C. to −90° C.
